(19) United States
(12) Patent Application Publication
(10) Pub. No.: US 2025/0318771 A1
(43) Pub. Date: Oct. 16, 2025

(54) IDENTIFICATION AND PROGNOSIS SYSTEM, OPERATION METHOD THEREOF AND NON-TRANSITORY COMPUTER READABLE MEDIUM

(71) Applicants: Taipei Medical University (TMU), Taipei City (TW); TAIPEI VETERANS GENERAL HOSPITAL, Taipei City (TW)

(72) Inventors: Syu-Jyun PENG, Zhubei City (TW); ShiauShian HUANG, Taipei City (TW)

(21) Appl. No.: 19/005,976

(22) Filed: Dec. 30, 2024

(30) Foreign Application Priority Data
Apr. 15, 2024 (TW) 113114024

(51) Int. Cl. A61B 5/374; A61B 5/00

(52) U.S. Cl. CPC A61B 5/374; A61B 5/726; A61B 5/7264

(57) ABSTRACT

The present disclosure provides an operating method of an identification and prognosis system, which includes steps as follows. The pre-process is performed on the EEG to obtain the pre-processed EEG; the pre-processed EEG is split into a plurality of different frequency band EEGs; a variety of brain functional connectivity features and a variety of power features are extracted from the different frequency band EEGs; the variety of brain functional connectivity features and the variety of power features are used for a machine learning to obtain an identification and prognosis model.

### RELATED APPLICATION

This application claims priority to Taiwan Patent Application No. 113114024, filed on Apr. 15, 2024, the entirety of which is hereby incorporated by reference.

### BACKGROUND

### Field of Invention

The present invention relates to systems and operation methods, particularly identification and prognosis systems and operation methods thereof.

### Description of Related Art

Depression is a physical and mental disease that seriously affects an individual's quality of life and poses a significant burden to the social economy. The disease presents diverse symptoms, such as changes in depression, sleep and appetite, and fluctuations in mental state. Patients are often accompanied by irrational feelings of guilt and suicidal thoughts, and extreme cases may lead to self-harm or suicide. In current clinical practice, drug treatment is still the main strategy to combat depression. Commonly used drugs are selective serotonin reuptake inhibitors (SSRI) and serotonin-norepinephrine reuptake inhibitors (SNRI). However, the treatment effects of many patients are not as good as expected. There is still a lack of clinically reliable biomarkers to diagnose or predict the efficacy of drugs. Treatment progress mostly relies on physicians' empirical judgment and patients' subjective descriptions.

In addition, the reaction period from the beginning of medication to its effectiveness is a period of many challenges for patients. Studies have pointed out that only 40 to 60% of patients achieve remission after undergoing two different drug treatments, and the remaining patients are classified as a group that is difficult to treat with drug treatment. When the first-choice drug fails to produce the desired effect, patients may become impatient, discontinue treatment, and suffer from symptoms of the disease. At the economic level, refractory patients often have to bear higher personal and medical economic costs. Therefore, if a patient's response to a specific drug can be predicted at the early stage of treatment, it will not only help shorten the time for testing different drugs, but also reduce the financial burden on patients.

### SUMMARY

In one or more various aspects, the present disclosure is directed to identifying and quantizing systems and operation methods thereof.

An embodiment of the present disclosure is related to an identification and prognosis system. The identification and prognosis system includes a storage device and a processor. The storage device is configured to store at least one instruction. The processor is coupled to the storage device, and the processor is configured to access and execute the at least one instruction for: performing a pre-process on an electroencephalogram (EEG) to obtain a pre-processed EEG; splitting the pre-processed EEG into a plurality of different frequency band EEGs; extracting a variety of brain functional connectivity features and a variety of power features from the plurality of the different frequency band EEGs; and using the variety of the brain functional connectivity features and the variety of the power features for a machine learning to obtain an identification and prognosis model.

In one embodiment of the present disclosure, the pre-process executed by the processor includes: performing a bandpass filtering on the EEG to obtain a 0.5-50 Hz band EEG; re-referencing the 0.5-50 Hz frequency band EEG to remove common components between different channels in the 0.5-50 Hz frequency band EEG, thereby obtaining a re-referencing EEG; and performing an independent component analysis on the re-referencing EEG to remove an eye movement signal from the re-referencing EEG, thereby obtaining the pre-processed EEG.

In one embodiment of the present disclosure, the processor accesses and executes the at least one instruction for: filtering the pre-processed EEG to obtain a delta band EEG, a theta band EEG, an alpha band EEG and a beta band EEG.

In one embodiment of the present disclosure, the processor accesses and executes the at least one instruction for: extracting a phase locking value between each two channels in each frequency band EEG from the plurality of the different frequency band EEGs; and extracting a phase lag index and a weighted phase lag index between the each two channels in the each frequency band EEG from the plurality of the different frequency band EEGs, where the variety of the brain functional connectivity features includes the phase locking value, the phase lag index and the weighted phase lag index.

In one embodiment of the present disclosure, the processor accesses and executes the at least one instruction for: performing a wavelet transformation on each channel in each frequency band EEG extracted from the plurality of the different frequency band EEGs to obtain an absolute power and a relative power of the each channel, where the variety of the power features includes the absolute power and the relative power.

Another embodiment of the present disclosure is related to an operation method of an identification and prognosis system. The operation method includes steps of: (A) performing a pre-process on an electroencephalogram (EEG) to obtain a pre-processed EEG; (B) splitting the pre-processed EEG into a plurality of different frequency band EEGs; (C) extracting a variety of brain functional connectivity features and a variety of power features from the different frequency band EEGs; and (D) using the variety of brain functional connectivity features and the variety of power features for a machine learning to obtain an identification and prognosis model.

In one embodiment of the present disclosure, the step (A) includes: performing a bandpass filtering on the EEG to obtain a 0.5-50 Hz band EEG; re-referencing the 0.5-50 Hz frequency band EEG to remove common components between different channels in the 0.5-50 Hz frequency band EEG, thereby obtaining a re-referencing EEG; and performing an independent component analysis on the re-referencing EEG to remove an eye movement signal from the re-referencing EEG, thereby obtaining the pre-processed EEG.

In one embodiment of the present disclosure, the step (B) includes: filtering the pre-processed EEG to obtain a delta band EEG, a theta band EEG, an alpha band EEG and a beta band EEG.

In one embodiment of the present disclosure, the step (C) includes: extracting a phase locking value between each two channels in each frequency band EEG from the plurality of the different frequency band EEGs; extracting a phase lag index and a weighted phase lag index between the each two channels in the each frequency band EEG from the plurality of the different frequency band EEGs, where the variety of the brain functional connectivity features includes the phase locking value, the phase lag index and the weighted phase lag index; and performing a wavelet transformation on each channel in the each frequency band EEG extracted from the plurality of the different frequency band EEGs to obtain an absolute power and a relative power of the each channel, where the variety of the power features includes the absolute power and the relative power.

In one embodiment of the present disclosure, the step (D) includes: inputting the phase locking value, the phase lag index and the weighted phase lag index between the each two channels in the each frequency band EEG of the plurality of the different frequency band EEGs and the absolute power and the relative power of the each channel in the each frequency band EEG of the plurality of the different frequency band EEGs to a plurality of different classifiers for training and verification of the machine learning, and after the machine learning, selecting a classifier with a highest evaluation index from the plurality of the different classifiers to be the identification and prognosis model.

Technical advantages are generally achieved, by embodiments of the present disclosure. Through the identification and prognosis system and its operation method of the present disclosure, the identification and prognosis model can automatically interpret and analyze EEG to obtain diagnosis of brain-related problems and/or treatment prognosis assessment. With the assistance of the identification and prognosis model, clinicians can not only quickly understand the current status of the patient's brain function before treatment, capable of assisting in the diagnosis of depression, but also can provide objective quantitative analysis on the improvement of brain function before and after treatment, to improve the accuracy and timeliness of treatment.

Many of the attendant features will be more readily appreciated, as the same becomes better understood by reference to the following detailed description considered in connection with the accompanying drawings.

### BRIEF DESCRIPTION OF THE DRAWINGS

The invention can be more fully understood by reading the following detailed description of the embodiment, with reference made to the accompanying drawings as follows:

FIG. 1 is a block diagram of an identification and prognosis system according to one embodiment of the present disclosure; and

FIG. 2 is a flow chart of an operation method of the identification and prognosis system according to one embodiment of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

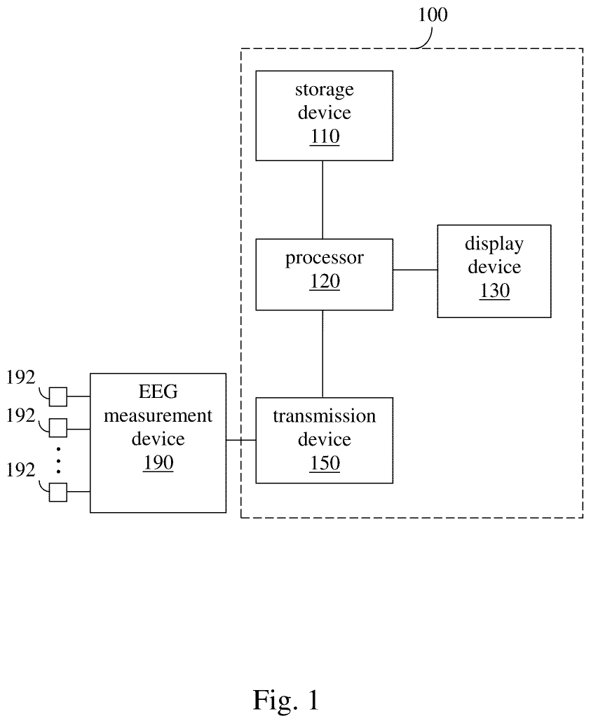

Referring to FIG. 1, in one aspect, the present disclosure is directed to a identification and prognosis system 100. The identification and prognosis system 100 may be easily integrated into a computer and may be applicable or readily adaptable to all technologies. Technical advantages are generally achieved by the identification and prognosis system 100 according to embodiments of the present disclosure. Herewith the identification and prognosis system 100 is described below with FIG. 1.

The subject disclosure provides the identification and prognosis system 100 in accordance with the subject technology. Various aspects of the present technology are described with reference to the drawings. In the following description, for purposes of explanation, numerous specific details are set forth in order to provide a thorough understanding of one or more aspects. It can be evident, however, that the present technology can be practiced without these specific details. In other instances, well-known structures and devices are shown in block diagram form in order to facilitate describing these aspects. The word “exemplary” is used herein to mean “serving as an example, instance, or illustration.” Any embodiment described herein as “exemplary” is not necessarily to be construed as preferred or advantageous over other embodiments.

In practice, for example, the identification and prognosis system 100 can be a computer server. The computer server can be remotely managed in a manner that substantially provides accessibility, consistency, and efficiency. Remote management removes the need for input/output interfaces in the servers. An administrator can manage a large data centers containing numerous rack servers using a variety of remote management tools, such as simple terminal connections, remote desktop applications, and software tools used to configure, monitor, and troubleshoot server hardware and software.

As used herein, “around”, “about”, “substantially” or “approximately” shall generally mean within 20 percent, preferably within 10 percent, and more preferably within 5 percent of a given value or range. Numerical quantities given herein are approximate, meaning that the term “around”, “about”, “substantially” or “approximately” can be inferred if not expressly stated.

In practice, in an embodiment of the present disclosure, the identification and prognosis system 100 can selectively establish a connection with the EEG measurement device 190. It should be understood that in the embodiments and the scope of the patent application, the description involving “connection” can generally refer to a component that indirectly communicates with another component by wired and/or wireless communication through another component, or a component that is physically connected to another element without through another element. For example, the identification and prognosis system 100 can indirectly communicate with the EEG measurement device 190 through wired and/or wireless communication via another component, or the identification and prognosis system 100 can be physically connected to the EEG measurement device 190 without another component. Those with ordinary skill in the art may select the connection manner depending on the desired application.

FIG. 1 is a block diagram of the identification and prognosis system 100 in infants according to one embodiment of the present disclosure. As shown in FIG. 1, the identification and prognosis system 100 includes a storage device 110, a processor 120, a transmission device 150 and a display device 130. For example, the storage device 110 can be a hard drive, a flash memory or another storage device, the processor 120 can be a central processing unit, the display device 130 can be a built-in display or an external screen, and the transmission device 150 can be a connector, a wired and/or wireless network device or another transmission interface.

In structure, the identification and prognosis system 100 is electrically connected to the EEG measurement device 190, the storage device 110 is electrically connected to the processor 120, the processor 120 is electrically connected to the display device 130, and the transmission device 150 is electrically connected to the processor 120. It should be noted that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. For example, the storage device 110 may be a built-in storage device that is directly connected to the processor 120, or the storage device 110 may be an external storage device that is indirectly connected to the processor 120 through the network device.

In practice, for example, the EEG measurement device 190 can measure an electroencephalogram (EEG). In practice, for example, the EEG measurement device 190 can measure EEG through multiple electrodes 192, so that the EEG includes data from multiple channels corresponding to multiple electrodes 192 to reflect the status of multiple brain regions. Although only one EEG measurement device 190 is shown in FIG. 1, this does not limit the present disclosure. In practice, EEG measurement device 190 can generally refer to one or more EEG measurement devices. Those skilled in the art can flexibly choose one or more number of EEG measurement devices.

In some embodiments of the present disclosure, the storage device 110 stores the EEG and at least one instruction, and the processor 120 is configured to access and execute the at least one instruction for: performing a pre-process on an electroencephalogram (EEG) to obtain a pre-processed EEG; splitting the pre-processed EEG into a plurality of different frequency band EEGs; extracting a variety of brain functional connectivity features and a variety of power features from the plurality of the different frequency band EEGs; and using the variety of the brain functional connectivity features and the variety of the power features for a machine learning to obtain an identification and prognosis model.

In use, the identification and prognosis model can automatically interpret and analyze EEG to obtain a diagnosis and/or treatment prognosis assessment of brain-related problems; for example, the content of diagnosis and/or treatment prognosis assessment may include: comparing the differences in brain network characteristics between patients and the general population or the changes in brain network characteristics before and after treatment. The display device 130 can display content of diagnosis and/or treatment prognosis assessment. With the assistance of the identification and prognosis model, clinicians can not only quickly find the lesions that need treatment without omissions, but also estimate the future efficacy of the changes in network characteristics before and after initial treatment, thereby improving the accuracy and security of treatment.

Regarding the specific mechanism of the above-mentioned pre-process, in some embodiments of the present disclosure, the pre-process executed by the processor 120 includes: performing a bandpass filtering on the EEG to obtain a 0.5-50 Hz band EEG, thereby removing noise; re-referencing the 0.5-50 Hz frequency band EEG to remove common components between different channels in the 0.5-50 Hz frequency band EEG, thereby obtaining a re-referencing EEG to reflect the real intensities of the brain waves of different channels; and performing an independent component analysis on the re-referencing EEG to remove an eye movement signal from the re-referencing EEG, thereby obtaining the pre-processed EEG that is beneficial to subsequent machine learning.

Regarding the specific mechanism of the above different frequency band EEGs, in some embodiments of the present disclosure, the processor 120 accesses and executes the at least one instruction for: filtering the pre-processed EEG to obtain a delta band (0.5-4 Hz) EEG, a theta band (4-8 Hz) EEG, an alpha band (8-13 Hz) EEG and a beta band (13-30 Hz) EEG, to reflect different states of the brain.

Regarding the specific mechanisms of the above variety of brain functional connectivity features, in some embodiments of the present disclosure, the processor 120 accesses and executes the at least one instruction for: extracting a phase locking value (PLV) between each two channels in each frequency band EEG from the plurality of the different frequency band EEGs; and extracting a phase lag index (PLI) and a weighted phase lag index (Wolli) between the each two channels in the each frequency band EEG from the plurality of the different frequency band EEGs, where the variety of the brain functional connectivity features includes the phase locking value, the phase lag index and the weighted phase lag index. In practice, the phase locking value reflects the phase synchronization of the signals between the two channels in the frequency band EEG, and the phase lag index and weighted phase lag index reflect the phase angle delay between the two channels, and the phase locking value, the phase lag index and the weighted phase lag index are beneficial to subsequent machine learning.

Regarding the specific mechanism of the above variety of power features, in some embodiments of the present disclosure, the processor 120 accesses and executes the at least one instruction for: performing a wavelet transformation on each channel in each frequency band EEG extracted from the plurality of the different frequency band EEGs to obtain an absolute power and a relative power of the each channel, where the variety of the power features includes the absolute power and the relative power. In practice, the absolute power and the relative power reflect the state of the brain area corresponding to each channel in the frequency band EEG, and the absolute power and the relative power are beneficial to subsequent machine learning.

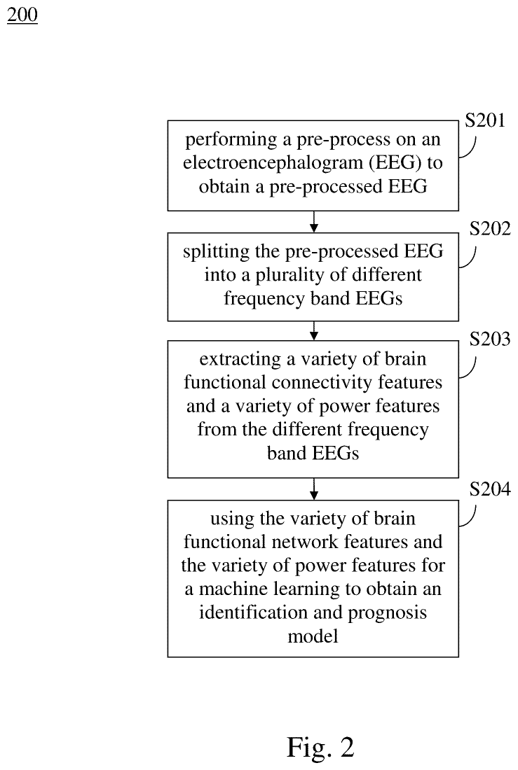

For a more complete understanding of an operation method of the a identification and prognosis system 100, referring FIGS. 1-2, FIG. 2 is a flow chart of the operation method 200 of the identification and prognosis system 100 according to one embodiment of the present disclosure. As shown in FIG. 2, the operation method 200 includes operations S201-S204. However, as could be appreciated by persons having ordinary skill in the art, for the steps described in the present embodiment, the sequence in which these steps are performed, unless explicitly stated otherwise, can be altered depending on actual needs; in certain cases, all or some of these steps can be performed concurrently.

The operation method 200 may take the form of a computer program product on a computer-readable storage medium having computer-readable instructions embodied in the medium. Any suitable storage medium may be used including non-volatile memory such as read only memory (ROM), programmable read only memory (PROM), erasable programmable read only memory (EPROM), and electrically erasable programmable read only memory (EEPROM) devices; volatile memory such as SRAM, DRAM, and DDR-RAM; optical storage devices such as CD-ROMs and DVD-ROMs; and magnetic storage devices such as hard disk drives and floppy disk drives.

In some embodiments of the present disclosure, in step S201, a pre-process is performed on an electroencephalogram (EEG) to obtain a pre-processed EEG; in step S202, the pre-processed EEG is split into a plurality of different frequency band EEGs; in step S203, a variety of brain functional connectivity features and a variety of power features are extracted from the different frequency band EEGs; in step S204, the variety of brain functional connectivity features and the variety of power features are used for a machine learning to obtain an identification and prognosis model.

Regarding the EEG data set, for example, a total of 46 healthy subjects and 77 patients with depression were compiled through historical data. The average age was 40.98±17.26 years old, and the male to female ratio was 28.46%: 71.54%. There was no statistically significant difference in age and gender between the major depressive disorder (MDD) group and the healthy group. In contrast, the MDD group was less likely to be employed and had less religious or exercise habits (p<0.05). This disclosure collects the EEG of patients before they receive antidepressant treatment (all patients are in a resting state with their eyes closed), and records the patient's response before receiving antidepressant treatment and after receiving antidepressant treatment for 4, 6 and 8 weeks. Response to treatment for depression was defined as change in score on the Hamilton depression scale (HAMD). The patient's HAMD score before receiving depression treatment was used as the basis of the patient's condition, and changes in the HAMD score were recorded after 4, 6, and 8 weeks of depression treatment. If the HAMD score in that week was reduced by 50% compared with the HAMD score before treatment, it was once called the treatment improvement group. On the other hand, if the HAMD score does not decrease by 50% compared with the HAMD score before treatment, it is defined as the non-improvement group. Among the 77 MDD patients, 54 patients were classified as the non-improvement group and 23 patients were in the treatment improvement group at week four. The proportion of patients in the treatment improvement group who used Benzodiazepine (BZD) was smaller than that in the treatment non-response group (p<0.05). In the eighth week, there were 37 people in the improvement group and 40 people in the non-improvement group. There were no statistically significant differences in age, gender, and educational background between the two groups. The EEG was recorded with the subject at rest with eyes closed at a sampling rate of 256 Hz. The electrodes 192 can be a total of 19 gold-silver electrodes (Neuroscan Inc.), according to the standard position of the 10-20 system, the conductive paste is applied to the skin, data are continuously recorded on a 32-channel EEG machine (Natus Nicolet One vEEG), and EEG impedance values are maintained below 10 KW.

In some embodiments of the present disclosure, the step S201 includes: performing a bandpass filtering on the EEG to obtain a 0.5-50 Hz band EEG; re-referencing the 0.5-50 Hz frequency band EEG to remove common components between different channels in the 0.5-50 Hz frequency band EEG, thereby obtaining a re-referencing EEG; and performing an independent component analysis on the re-referencing EEG to remove an eye movement signal from the re-referencing EEG, thereby obtaining the pre-processed EEG.

In practice, for example, EEG data is imported into the EEGLAB v2019.0 open toolbox based on MATLAB to implement the above-mentioned pre-process. Because the average intensity of different brainwave channels is different, the above-mentioned re-referencing can remove common components between different channels. The different independent components (IC) are separated through the independent component analysis (ICA) to remove the eye movement signal.

In some embodiments of the present disclosure, the step S202 includes: filtering the pre-processed EEG to obtain a delta band EEG, a theta band EEG, an alpha band EEG and a beta band EEG.

In some embodiments of the present disclosure, the step S203 includes: extracting a phase locking value (PLV) between each two channels in each frequency band EEG from the plurality of the different frequency band EEGs; extracting a phase lag index (PLI) and a weighted phase lag index (wPLI) between the each two channels in the each frequency band EEG from the plurality of the different frequency band EEGs, where the variety of the brain functional connectivity features includes the phase locking value, the phase lag index and the weighted phase lag index; and performing a wavelet transformation on each channel in the each frequency band EEG extracted from the plurality of the different frequency band EEGs to obtain an absolute power and a relative power of the each channel, where the variety of the power features includes the absolute power and the relative power.

Regarding the extraction mechanism of brain functional connectivity features of EEG, in practice, for example, a total of three phase synchronizations are used to establish the brain functional network, namely the phase locking value (PLV), the phase lag index (PLI) and the weighted phase lag index (wPLI).

The delta, theta, alpha and beta bands for all electrodes 192 (such as: Fp1, Fp2, F3, F4, C3, C4, P3, P4, O1, O2, F7, F8, T3, T4, T5, T6, Fz, Cz and Pz) with PLV, PLI, and wPLI are used to calculate the functional connection strength, so that the functional network among a wide range of brain regions can be obtained.

The process of establishing and quantifying brain functional networks is as follows. The first step is to calculate 19×19 PLV, PLI and wPLI functional network matrixes respectively from the 2nd second to the 58th second of the pre-processed EEG. The second step is to extract the upper triangular part of the functional network matrix (excluding diagonal elements) and arrange the upper triangular part into a 1×171 vector. The third step is to combine the PLV, PLI and wPLI connection strengths of above four frequency bands to get a 1×684 vector.

The formula of PLV is expressed as

$$PLV = \vert \frac{1}{N} {\sum }_{k = 1}^{N} {e}^{i ( {φ}_{l} ( k ) - {φ}_{m} ( k ) )} \vert .$$

The PLV can be used to calculate the phase synchronization of the two-channel signals. When there are two EEG channels I and m, the PLV value between the two channels can be calculated through the above formula. The PLV value is between 0-1. The value is closer to 1, the phases of the two signals are more synchronized.

The formula of PLI is expressed as

$$PLI = \vert \frac{1}{N} {\sum }_{k = 1}^{N} sgn ( [ {e}^{{i ( {θ}^{j} - {θ}^{k} )}_{t}} ] ) \vert .$$

The formula of wPLI is expressed as

$$wPLI = \frac{\vert \frac{1}{N} {\sum }_{k = 1}^{N} \vert ( {S}_{k} ) \vert sgn ( [ {s}_{k} ] ) \vert}{\frac{1}{N} {\sum }_{k = 1}^{N} \vert ( {S}_{k} ) \vert} .$$

PLI and wPLI can identify the phase angle delay between the two channels. The value of PLI is between 0 and 1. When PLI is 1, it means there is a fixed phase angle delay between the two channels. PLI is very sensitive to noise. If the phase angle changes slightly, the sign of the phase difference may change. Therefore, in order to solve this problem, WPLI takes the amplitude into consideration.

Regarding the extraction mechanism of the absolute/relative power feature of EEG, in practice, for example, the present disclosure can perform continuous wavelet with transformation time-frequency analysis on EEG. Unlike Fourier transform, which uses infinitely continuous sine and cosine functions to decompose signals, the wavelet analysis uses wavelet functions of finite length and satisfying

$${\int }_{- ∞}^{∞} ψ ( t ) dt = 0$$

as the basis for signal decomposition. This function is called a wavelet function. Wavelet theory translates and expands or compresses the wavelet function and uses different weighting values to reconstruct the signal. The process of wavelet transformation is to find the corresponding weight value ψ(τ, s) under various translations (the displacement is represented by τ) and expansion or compression (controlled by the variable s). The mathematical expression of continuous wavelet transformation is:

$$ψ ( τ , s ) = WT \{ x ( t ) \} = \frac{1}{\sqrt{s ∨} {\int }_{- ∞}^{∞} {ψ}^{*} ( \frac{t - τ}{s} ) dt} ,$$

where * represents complex conjugate, and s is a scaling factor. When s<1, the continuous wavelet transformation compresses; T is a translation parameter of controlling the left and right movement of the wavelet. The wavelet transformation is essentially a time proportional analysis method. At smaller (or larger) scale factors, the wavelet function becomes narrower (or wider), corresponding to higher (or lower) frequencies. This disclosure can use continuous wavelet transformation to extract EEG power characteristics of different frequency bands, by using two types of power quantification, namely the absolute power and the relative power. After calculating the power intensity of the delta, theta, alpha and beta frequency bands of all electrodes, the absolute power and relative power of each channel can be obtained.

The process of calculating power is as follows. The first step is to calculate the absolute power and relative power of each channel using the 2nd to 58th second of the pre-processed EEG; the second step is to string the absolute powers and the relative power of different frequency bands, and they are combined into a 1×76 vector.

In some embodiments of the present disclosure, the step S204 includes: inputting the phase locking value, the phase lag index and the weighted phase lag index between the each two channels in the each frequency band EEG of the plurality of the different frequency band EEGs and the absolute power and the relative power of the each channel in the each frequency band EEG of the plurality of the different frequency band EEGs to a plurality of different classifiers for training and verification of the machine learning, and after the machine learning, selecting a classifier with a highest evaluation index from the plurality of the different classifiers to be the identification and prognosis model.

Regarding the above-mentioned multiple different classifiers, in practice, for example, the present disclosure uses machine learning to train the model, and the five feature vectors (PLV, PLI, wPLI, absolute power and relative power) for ten different classifiers are trained and verified, as detailed below.

(1) Decision Tree: the decision tree is a machine learning model for supervised learning, often used for classification and regression tasks. It splits data according to feature values and divides the data set into different blocks, where each block represents a decision path. These segmentation processes are based on different features and their thresholds, so that the branches of the tree are classified according to the data characteristics, and finally form leaf nodes, which represent the prediction results of the branch. Some embodiments of the present disclosure use a fine tree, a medium tree and a coarse tree. The difference between these three decision trees is the maximum number of branches. The maximum number of branches of the fine tree is 100, and the maximum number of branches of medium tree is 20, and the maximum number of branches the rough tree is 4. With a larger number of branches, the ability to fit the training data is stronger, but it is also prone to over-fitting problems; with a smaller number of branches, although the fitting ability on the training data may be poor, the strong ability for generalizing to new data can reduce the risk of overfitting.

(2) Discriminant Analysis: the discriminant analysis is a statistical method used to classify data into two or more groups. Its main purpose is to find linear combinations of one or more variables that have the greatest variability among different groups and the smallest variability within the same group. The goal of this approach is to maximize variability between different groups and minimize variability within the same group. Some embodiments of the present disclosure use the linear discriminant analysis (LDA), which is a common form of discriminant analysis and is mainly used for dimensionality reduction and classification.

(3) Logistic Regression Classifiers: the logistic regression classifier is a commonly used statistical learning method, used to solve binary classification problems. A logistic function converts the linear combinations of input features into a probability value between 0 and 1 to predict the likelihood of belonging to a certain category. Some embodiments of the present disclosure use a binary GLM logistic regression and an efficient logistic regression; the former has a wider range of applications and can perform different types of classification tasks, and the latter focuses on improving the efficiency of the model to make it more suitable for large data sets or scenarios that require faster training.

(4) Naive Bayes Classifiers: the naive Bayes classifiers is a classification algorithm based on Bayes theorem and the assumption of conditional independence between features. This algorithm assumes that all features are independent of each other for a given category, that is, the presence of one feature is independent of the presence of other features. Some embodiments of the present disclosure use a Gaussian naive Bayes and a kernel naive Bayes; the former is suitable for continuous features, and the latter is suitable for nonlinear problems.

(5) Support Vector Machines (SVM): the upport vector machine is a powerful supervised machine learning algorithm used for classification and regression tasks. The main goal is to find an optimal hyperplane (for linear SVM), or decision boundary, that can effectively separate data points of different categories and can maximize the margin, that is, the minimum distance from the data point to the hyperplane. The SVM performs well in high-dimensional space, and non-linear classification problems can be handled through kernel techniques. Some embodiments of the present disclosure use a linear support vector machine (Linear SVM), a quadratic support vector machine (Quadratic SVM), a cubic support vector machine (Cubic SVM), a fine Gaussian support vector machine (Fine Gaussian SVM), a medium Gaussian support SVM and a Coarse Gaussian SVM.

(6) Efficiently Trained Linear Classifiers: The efficiently trained linear classifiers refer to linear models trained in a more efficient manner. These models are often optimized for large data sets or situations where fast training is required. The goal is to increase training speed and reduce resource consumption while maintaining or improving the performance of the model. This optimization may involve improving the training algorithm, using more efficient optimization techniques, or reducing computational costs to speed up the model training process. Some embodiments of the present disclosure use an Efficient Linear Support Vector Machine (Efficient Linear SVM), which is an optimized linear support vector machine model. It focuses on improving the training efficiency of linear SVM, making it more suitable for large data sets or scenarios that require faster training. This optimization may include using faster optimization algorithms, improving computational processes, or finding more efficient feature representations to improve model efficiency and performance.

(7) Nearest Neighbor Classifiers: The nearest neighbor classifier is a supervised machine learning algorithm based on the proximity metric method, used for classification problems. It classifies data points based on distance or similarity between them. When a new data point needs to be classified, the nearest neighbor classifier finds the nearest neighbor to that point in the training data and makes a prediction based on its category. Some embodiments of the disclosure use a Fine KNN, a Medium KNN, a Coarse KNN, a Cosine KNN, a Cubic KNN and a Weighted KNN.

(8) Kernel Approximation Classifiers: The kernel approximation classifiers are classifiers that use kernel approximation methods to accelerate calculations. They aim to approximate a high-dimensional kernel function into a low-dimensional version to reduce computational complexity, especially for large data sets and high-dimensional feature spaces. Some embodiments of the present disclosure uses support a SVM kernel and a Logistic Regression kernel.

(9) Ensemble Classifiers: The ensemble classifier is a method of improving prediction performance by combining multiple basic classifiers. Some embodiments of the present disclosure use boosted trees, bagged trees, subspace discriminant, a subspace KNN and RUSBoosted trees.

(10) Neural Network Classifiers: the neural network classifier is a machine learning model that uses a multi-layer structure composed of neurons for classification. They are based on multi-layered neural networks and are trained to learn complex patterns and features. Neural networks with different layers and widths show different model structures and capabilities. Some embodiments of the present disclosure use a narrow neural network, a medium neural network, a wide neural network, a bi-layered neural network and a tri-layered neural Network.

Regarding the above machine learning, in practice, for example, the model training of the machine learning of the present disclosure adopts the leave-one-out cross-validation (LOOCV) method; The LOOCV refers to one of the data is used as verification data, and the remaining data is used as training data. The training is repeated until each sample is used as the verification data once.

Regarding the above evaluation index, in practice, for example, the evaluation index can be a precision, a recall (also known as sensitivity), specificity, accuracy and/or a F1-score.

In practical applications, for example, the precision refers to the proportion of actually effectively treated patients to predictively effectively treated patients predicted by the identification and prognosis model; the recall refers to a proportion of the predictively effectively treated patients predicted by the identification and prognosis model in the actually effectively treated patients to the actually effectively treated patients; the sensitivity refers to a proportion of actually ineffectively treated patients to predictively ineffectively treated patients predicted by the identification and prognosis model; the accuracy refers a correctly predictive proportion through the identification and prognosis model.

In view of the above, technical advantages are generally achieved, by embodiments of the present disclosure. Through the identification and prognosis system 100 and its operation method 200 of the present disclosure, the identification and prognosis model can automatically interpret and analyze EEG to obtain diagnosis of brain-related problems and/or treatment prognosis assessment. With the assistance of the identification and prognosis model, clinicians can not only quickly and without omissions find the lesions that need treatment (such as depression), but also quantitatively analyze the possible efficacy of different therapies before and after treatment, thereby improving Treatment accuracy and safety.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An identification and prognosis system, comprising:
a storage device configured to store at least one instruction; and
a processor coupled to the storage device, and the processor configured to access and execute the at least one instruction for:
performing a pre-process on an electroencephalogram (EEG) to obtain a pre-processed EEG;
splitting the pre-processed EEG into a plurality of different frequency band EEGs;
extracting a variety of brain functional connectivity features and a variety of power features from the plurality of the different frequency band EEGs; and
using the variety of the brain functional connectivity features and the variety of the power features for a machine learning to obtain an identification and prognosis model.

2. The identification and prognosis system of claim 1, wherein the pre-process executed by the processor comprises:
performing a bandpass filtering on the EEG to obtain a 0.5-50 Hz band EEG;
re-referencing the 0.5-50 Hz frequency band EEG to remove common components between different channels in the 0.5-50 Hz frequency band EEG, thereby obtaining a re-referencing EEG; and
performing an independent component analysis on the re-referencing EEG to remove an eye movement signal from the re-referencing EEG, thereby obtaining the pre-processed EEG.

3. The identification and prognosis system of claim 1, wherein the processor accesses and executes the at least one instruction for:
filtering the pre-processed EEG to obtain a delta band EEG, a theta band EEG, an alpha band EEG and a beta band EEG.

4. The identification and prognosis system of claim 1, wherein the processor accesses and executes the at least one instruction for:
extracting a phase locking value between each two channels in each frequency band EEG from the plurality of the different frequency band EEGs; and
extracting a phase lag index and a weighted phase lag index between the each two channels in the each frequency band EEG from the plurality of the different frequency band EEGs, wherein the variety of the brain functional connectivity features comprises the phase locking value, the phase lag index and the weighted phase lag index.

5. The identification and prognosis system of claim 1, wherein the processor accesses and executes the at least one instruction for:
performing a wavelet transformation on each channel in each frequency band EEG extracted from the plurality of the different frequency band EEGs to obtain an absolute power and a relative power of the each channel, wherein the variety of the power features comprises the absolute power and the relative power.

6. An operation method of an identification and prognosis system, and the operation method, comprising steps of:
(A) performing a pre-process on an electroencephalogram (EEG) to obtain a pre-processed EEG;
(B) splitting the pre-processed EEG into a plurality of different frequency band EEGs;
(C) extracting a variety of brain functional connectivity features and a variety of power features from the different frequency band EEGs; and
(D) using the variety of brain functional connectivity features and the variety of power features for a machine learning to obtain an identification and prognosis model.

7. The operation method of claim 6, wherein the step (A) comprises:
performing a bandpass filtering on the EEG to obtain a 0.5-50 Hz band EEG;
re-referencing the 0.5-50 Hz frequency band EEG to remove common components between different channels in the 0.5-50 Hz frequency band EEG, thereby obtaining a re-referencing EEG; and
performing an independent component analysis on the re-referencing EEG to remove an eye movement signal from the re-referencing EEG, thereby obtaining the pre-processed EEG.

8. The operation method of claim 6, wherein the step (B) comprises:
filtering the pre-processed EEG to obtain a delta band EEG, a theta band EEG, an alpha band EEG and a beta band EEG.

9. The operation method of claim 6, wherein the step (C) comprises:
extracting a phase locking value between each two channels in each frequency band EEG from the plurality of the different frequency band EEGs;
extracting a phase lag index and a weighted phase lag index between the each two channels in the each frequency band EEG from the plurality of the different frequency band EEGs, wherein the variety of the brain functional connectivity features comprises the phase locking value, the phase lag index and the weighted phase lag index; and
performing a wavelet transformation on each channel in the each frequency band EEG extracted from the plurality of the different frequency band EEGs to obtain an absolute power and a relative power of the each channel, wherein the variety of the power features comprises the absolute power and the relative power.

10. The operation method of claim 9, wherein the step (D) comprises:
inputting the phase locking value, the phase lag index and the weighted phase lag index between the each two channels in the each frequency band EEG of the plurality of the different frequency band EEGs and the absolute power and the relative power of the each channel in the each frequency band EEG of the plurality of the different frequency band EEGs to a plurality of different classifiers for training and verification of the machine learning, and after the machine learning, selecting a classifier with a highest evaluation index from the plurality of the different classifiers to be the identification and prognosis model.

11. A non-transitory computer readable medium to store a plurality of instructions for commanding a computer to execute an operation method, and the operation method comprising steps of:
(A) performing a pre-process on an electroencephalogram (EEG) to obtain a pre-processed EEG;
(B) splitting the pre-processed EEG into a plurality of different frequency band EEGs;
(C) extracting a variety of brain functional connectivity features and a variety of power features from the different frequency band EEGs; and
(D) using the variety of brain functional connectivity features and the variety of power features for a machine learning to obtain an identification and prognosis model.

12. The non-transitory computer readable medium of claim 11, wherein the step (A) comprises:
performing a bandpass filtering on the EEG to obtain a 0.5-50 Hz band EEG;
re-referencing the 0.5-50 Hz frequency band EEG to remove common components between different channels in the 0.5-30 Hz frequency band EEG, thereby obtaining a re-referencing EEG; and
performing an independent component analysis on the re-referencing EEG to remove an eye movement signal from the re-referencing EEG, thereby obtaining the pre-processed EEG.

13. The non-transitory computer readable medium of claim 11, wherein the step (B) comprises:
filtering the pre-processed EEG to obtain a delta band EEG, a theta band EEG, an alpha band EEG and a beta band EEG.

14. The non-transitory computer readable medium of claim 11, wherein the step (C) comprises:
extracting a phase locking value between each two channels in each frequency band EEG from the plurality of the different frequency band EEGs;
extracting a phase lag index and a weighted phase lag index between the each two channels in the each frequency band EEG from the plurality of the different frequency band EEGs, wherein the variety of the brain functional connectivity features comprises the phase locking value, the phase lag index and the weighted phase lag index; and
performing a wavelet transformation on each channel in the each frequency band EEG extracted from the plurality of the different frequency band EEGs to obtain an absolute power and a relative power of the each channel, wherein the variety of the power features comprises the absolute power and the relative power.

15. The non-transitory computer readable medium of claim 14, wherein the step (D) comprises:
inputting the phase locking value, the phase lag index and the weighted phase lag index between the each two channels in the each frequency band EEG of the plurality of the different frequency band EEGs and the absolute power and the relative power of the each channel in the each frequency band EEG of the plurality of the different frequency band EEGs to a plurality of different classifiers for training and verification of the machine learning, and after the machine learning, selecting a classifier with a highest evaluation index from the plurality of the different classifiers to be the identification and prognosis model.
